(19) United States
(12) Patent Application Publication
(10) Pub. No.: US 2010/0021413 A1
(43) Pub. Date: Jan. 28, 2010

(54) Malodor Counteracting Compositions

(75) Inventors: Thomas McGee, Nyack, NY (US); Felix Flaschsmann, Duebendorf (CH); Markus Gautschi, Zeiningen (CH); Richard P. Sgaramella, Hoboken, NJ (US); Venkateswara Kumar Vedantam, Paramus, NJ (US)

Correspondence Address: PARFOMAK, ANDREW N.;NORRIS MCLAUGHLIN & MARCUS PA, 875 THIRD AVE, 8TH FLOOR, NEW YORK, NY, 10022, US

(73) Assignee: GIVAUDAN SA, Vernier (CH)

(21) Appl. No.: 12/513,539

(22) PCT Filed: Nov. 6, 2007
(86) PCT No.: PCT/CH2007/000547

§ 371 Date: May 5, 2009

Related U.S. Application Data
(60) Provisional application No. 60/864,616, filed on Nov. 7, 2006.

(51) Int. Cl. A61L 9/01

(52) U.S. Cl. 424/76.2; 424/76.1

(57) ABSTRACT

A malodor counteracting composition comprising at least one compound of formula (A)

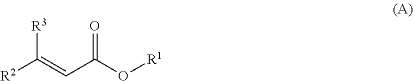

and at least one compound of formula (B)

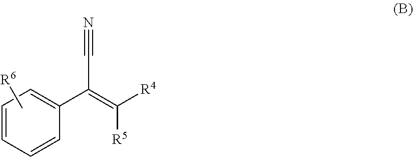

wherein R¹ to R⁶are as defined in the description.

The invention relates to malodor counteracting compositions. More particularly, the present invention relates to improved fragrance compositions containing two different classes of malodor counteractants, carboxylic acid esters and benzeneacetonitriles, respectively.

The problem of malodors has been with mankind for as long as civilization. There are very few articles or things which do not have some odor associated with them. Often this odor is undesirable, such as tobacco smoke odor, cooking odors, and odors of, e.g. mold, bathroom, pets etc. The compounds that cause malodor are often highly volatile, and are encountered in the air, as well as on substrates such as fabric, hard surfaces, skin and hair.

Most fragrance compositions merely mask undesirable odors with the stronger, desirable odor. In general, fragrance compositions do not in any way absorb or remove the odors but merely hide or mask the same.

There are several routes to reducing malodor, for example by using absorbers, such as sodium bicarbonate, activated charcoal, zinc ricinoleate, zeolites, ground corn cob cylodextrins. These compositions are only slightly effective and they have the disadvantage of removing both malodor and fragrance

Other approaches to reducing malodor that have been proposed include chemical neutralization reducing the partial vapor pressure of the malodor.

Whereas all of these approaches are partially effective, there is still an ongoing demand for even more efficient products, which may allow the use of lower concentrations of active compounds in an end product.

It has now been found that the combination of a known class of malodor counteractant molecules, as described, for example, in WO 02/051788 and U.S. Pat. No. 4,305,930, with a selected class of benzeneacetonitriles results in very efficient malodor counteractant compositions. Surprisingly, the specific combination of the two classes of molecules, as described hereinunder, results in an unexpected synergistic increase in counteracting capabilities.

Accordingly, the present invention refers in one of its aspects to a composition comprising
a) at least one compound of formula (A)

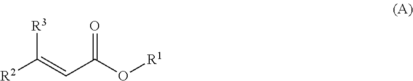

wherein
R¹ is linear or branched C₁-C₁₂ alkyl, e.g. ethyl and hexyl, linear or branched C₃-C₁₂ alkenyl, e.g. citronellyl, C₆-C₁₁ aryl, e.g. phenyl, C₇-C₁₂ arylalkyl, e.g. benzyl, or C₇-C₁₂ arylalkyl substituted with at least one O or N atom;
R² is linear or branched C₁-C₁₀ alkyl, e.g. methyl, ethyl and isopropyl, C₆-C₁₀ aryl, e.g. phenyl, C₇-C₁₀ alkoxyaryl, e.g. 4-methoxyphenyl, C₄-C₁₀ alkoxycarbonyl, e.g. hexyloxycarbonyl;
R³ is hydrogen or methyl; and
R² and R³ are either in the E or Z configuration with respect to the ester group; and
b) at least one compound of formula (B)

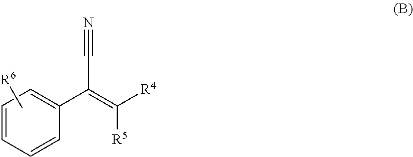

wherein
R⁴ and R⁵ are independently H, linear or branched C₁ to C₈ alkyl, e.g. methyl, ethyl, propyl, iso-butyl, n-butyl, tert-pentyl, iso-pentyl, and n-pentyl, or linear or branched C₂ to C₈ alkenyl, e.g. allyl and 1-butenyl, with the proviso that at least one of the residues R⁴ and R⁵ is not hydrogen; or
R⁴ and R⁵ form together with the carbon atom to which they are attached a 5 or 6 membered carbocycle, the carbocycle is optionally substituted with one methyl group;
R⁶is H, methyl, ethyl, propyl or isopropyl; and
the sum of carbon atoms of the compound of formula (B) is from 11 to 20, e.g 12, 14, 15 or 16.

In a preferred embodiment the compounds of formula (A) may be selected from citronellyl methylcrotonate (=3,7-dimethyloct-6-enyl 3-methylbut-2-enoate), geranyl crotonate, dihexyl fumarate, benzyl cinnamate, phenyl cinnamate and 2-ethyl-hexyl-para-methoxy-cinnamate (=octyl methoxy cinnamate).

The compounds of formula (B) may be selected from 3-methyl-2-phenylbut-2-enenitrile (compound 1), 3-methyl-2-phenylpent-2-enenitrile (compound 2), 2-phenyloct-2-enenitrile (compound 3), 2-cyclohexylidene-2-o-tolylacetonitrile (compound 4), 2-cyclohexylidene-2-p-tolylacetonitrile (compound 5) and 2-cyclohexylidene-2-phenylacetonitrile (compound 6).

The composition as hereinabove described preferably comprises a mixture of compound(s) of formula (A) and compound(s) of formula (B) in a ratio of from about 1:99 to about 99:1, preferably 10:90 to 50:50, e.g. 20:80 (A:B).

The composition of the present invention may further comprise other malodor counteractants such as malodor neutralizers and malodor absorbers. By “malodor neutralizer” is meant a material or a mixture thereof that reacts with malodor compounds such as certain amines, thiols, and short chain aliphatic acids. They may be preferably selected from aldehydes, such as alkyl aldehydes, benzaldehyde and vanillin; and cycloalkyl tertiary alcohols, such as 4-cyclohexyl-4-methyl-2-pentanone. By “malodor absorbers” is meant any material of large surface area capable of absorbing malodor. Such malodor absorbers include, for example, molecular sieves, such as zeolites, silicas, aluminosilicates, cyclodextrins; activated charcoal, clays, dried citrus pulp, cherry pit extract, corncob, and mixtures thereof.

The composition of the present invention may further comprise ingredients that retard the rate of build-up of malodor caused by bacterial breakdown, for example, antimicrobial agents and enzyme inhibitors. Such antimicrobial agents and enzyme inhibitors include, for example, metal salts such as zinc citrate, zinc oxide, zinc pyrethiones, and octopirox; organic acids, such as sorbic acid, benzoic acid, and their salts; parabens, such as methyl paraben, ethyl paraben, propyl paraben, isopropyl paraben, butyl paraben, isobutyl paraben, benzyl paraben, and their salts; alcohols, such as benzyl alcohol, phenyl ethyl alcohol; boric acid; 2,4,4′-trichloro-2-hydroxy-diphenyl ether (Triclosan™); phenolic compounds, such as phenol, 2-methyl phenol, 4-ethyl phenol; essential oils such as rosemary, thyme, lavender, eugenol, geranium, tea tree, clove, lemon grass, peppermint, or their active components such as anethole, thymol, eucalyptol, farnesol, menthol, limonene, methyl salicylate, salicylic acid, terpineol, nerolidol, geraniol, and mixtures thereof.

Optionally, the composition as defined above, i.e. a composition comprising at least one compound of formula (A) and at least one compound of formula (B), may be used in combination with known odorant molecules. Such molecules are, for example, described in “Perfume and Flavor Materials of Natural Origin”, S. Arctander, Ed., Elizabeth, N.J., 1960; “Perfume and Flavor Chemicals”, S. Arctander, Ed., Vol. I & II, Allured Publishing Corporation, Carol Stream, USA, 1994. Preferred are odorant molecules which are known as so-called deodorant perfume components, as disclosed, for example, in U.S. Pat. No. 4,663,068 which are hereby incorporated by reference.

Auxiliary ingredients, such as solvents, dyes and antioxidants, may also be added to the composition of the present invention in art-recognised quantities.

The solvents for use in the invention may be polar, such as ethanol, isopropanol, diethyleneglycol monoethyl ether, dipropylene glycol, diethyl phthalate, triethyl citrate, and triethylene glycols, or non-polar, such as isopropyl myristate and isoparaffinic hydrocarbons. Polar and non-polar solvents may be combined. The ratio of polar and non-polar solvents may be selected to provide the right properties for application and rate of release of the compounds of formula (A) and formula (B), as hereinabove described.

The compositions of the present invention may be added to a wide variety of consumer products, such as household products, personal care products and cosmetics, both perfumed and perfume-free.

Household products which may comprise a composition according to the invention include detergent, surface cleaner, including hard surface cleaner, dishwasher deodorizing products, kitchen garbage deodorizing products, air freshener, softener, bleach, fabric refresher, scourer and cat litter. The list of household products is given by way of illustration and is not to be regarded as being in any way limiting.

Personal care products and cosmetics which may comprise a composition according to the invention include lotion, e.g. after-shave lotion, shampoo, conditioner, styling spray, mousse, gel, hair wipe, hair spray, hair pomade, shower gel and bath salt, hygiene product, deodorant, antiperspirant, vanishing creme, depilatory, talcum powder and catamenial. The list of personal care products and cosmetics is given by way of illustration and is not to be regarded as being in any way limiting.

Typically the products comprise from about 0.0001% to about 20% by weight, preferably about 0.001% to about 10% by weight, of at least one compound of formula (A) and at least one compound of formula (B) as hereinabove defined, based on the product. The effective amount depends upon the type of product into which the mixture is admixed. For example, if used in a fabric refresher it may be added to a fragrance composition at around 1% by weight which is then added to the product at around 0.1% by weight, i.e. the fabric refresher comprises about 0.001% by weight of the composition as hereinabove described. Or, in a liquid electrical air freshener composition it may be added at around 20% by weight based on the air freshener composition.

Accordingly, the present invention refers in a further aspect to a consumer product comprising an effective malodor-counteracting amount of a composition comprising at least one compound of formula (A) and at least one compound of formula (B).

Another aspect of the invention is a method of removing malodor from the air or from surfaces, comprising applying thereto an effective amount of a composition comprising at least one compound of formula (A) and at least one compound of formula (B) as hereinabove described.

The invention is now further described with reference to the following non-limiting examples. These examples are of the purpose of illustration only and it is understood that variations and modifications can be made by one skilled in the art.

In this application, the term “%” or “percent” shall mean percent by weight, unless the context clearly indicates otherwise.

### EXAMPLE 1

### Determination of the Malodor Reduction Potential

A 1 liter glass headspace collection jar had placed inside it a 25 ml stoppered glass container which contained 0.5 g of a test compound/mixture of compounds as indicated in Table 1. 10 ul of hexyl amine as a representative malodor was injected into the headspace jar. This was left for 15 minutes at 25° C. to equilibrate. One ml/minute of the headspace was drawn for one minute through a Tenax™ headspace trap. The trap was removed and analyzed to determine the initial concentration of the malodor. The glass stopper was removed from the container and the test component and the malodor were left in contact for 60 minutes at 25° C. One ml/minute of the headspace was drawn for one minute through a Tenax™ headspace trap to determine the amount of malodor remaining. The Tenax traps were thermally desorbed into an Agilent 6890 GC/MS to analyze the quantity of hexyl amine. The results are shown in Table 1 below.

The following compounds of formula (B) were evaluated in combination with citronellyl methylcrotonate (also known as Sinodor ex Quest International, Ashford, Kent), an effective malodor counteractant molecule against a wide variety of malodors such as axilla odor, tobacco smoke odor, onion odor etc:

3-Methyl-2-phenylbut-2-enenitrile=compound 1

3-Methyl-2-phenylpent-2-enenitrile=compound 2

2-Phenyloct-2-enenitrile=compound 3

2-Cyclohexylidene-2-o-tolylacetonitrile=compound 4

2-Cyclohexylidene-2-p-tolylacetonitrile=compound 5

2-Cyclohexylidene-2-phenylacetonitrile=compound 6

TABLE 1
Percent reduction of hexyl amine

| Compound/Compound mixture | % Reduction of hexyl amine |
|---|---|
| citronellyl methylcrotonate | 61.3 |
| 50% compound 1 | 89.2 |
| 50% citronellyl methylcrotonate | |
| 50% compound 2 | 81.7 |
| 50% citronellyl methylcrotonate | |
| 50% compound 3 | 77.4 |
| 50% citronellyl methylcrotonate | |
| 50% compound 4 | 71.1 |
| 50% citronellyl methylcrotonate | |
| 50% compound 5 | 75.9 |
| 50% citronellyl methy;crotonate | |
| 50% compound 6 | 74.4 |
| 50% citronellyl methylcrotonate | |

As can be seen from the results above the use of compounds of formula (B) in combination with citronellyl methylcrotonate, i.e a compound of formula (A), significantly increases effectiveness of the reduction of malodor.

### EXAMPLE 2

### Determination of the Malodor Reduction Potential of Different Compounds

Following the same procedure described in Example 1, compounds according to formula (A) were mixed with an equal amount of 3-methyl-2-phenylpent-2-enenitrile. 0.5 g of the resulting mixture was tested against 10 ul hexyl amine as a representative malodor. The results are given in Table 2, below.

TABLE 2
Percent reduction of hexyl amine

| Compound/Compound mixture | % Reduction of hexyl amine |
|---|---|
| Geranyl crotonate | 71.0 |
| 50% compound 2 | 90.9 |
| 50% geranyl crotonate | |
| Dihexyl fumarate | 78.1 |
| 50% compound 2 | 92.1 |
| 50% dihexyl fumarate | |
| Benzyl cinnamate | 62.1 |
| 50% compound 2 | 88.9 |
| 50% cenzyl cinnamate | |
| Octyl methyl cinnamate | 58.7 |
| 50% compound 2 | 88.1 |
| 50% octyl methyl cinnamate | |

As can be seen from the results above the use of a compound of formula (A) in combination with 3-methyl-2-phenylpent-2-enenitrile increases the effectiveness of these known malodor counteractant molecules of formula (A).

### EXAMPLE 3

### Sensory Panel Determination of the Malodor Reduction Potential of Different Mixtures

Test mixtures of a compound of formula (A) and a compound of formula (B) in the ratio as given in the table (10% ethanol solution) were prepared. 0.5 g of a 1% ethanol solution of synthetic axilla malodor was placed onto cotton pads. 0.4 g of the ethanol solution of the test mixtures, respectively, was placed on each axilla malodor treated cotton pad. One pad was left untreated as the control. Each pad was allowed to dry for 15 minutes. The test cotton pads were randomized, and an expert panel of 5 was used to determine the intensity of the malodor. Each panelist was asked to check a box that represented the strength of the axilla malodor using a Labeled Magnitude Scale (LMS) (Barry G. Green, Pamela Dalton, Beverly Cowart, Greg Shaffer, Krystyna Rankin and Jennifer Higgins. Evaluating the Labeled Magnitude Scale for Measuring Sensations of Taste and Smell. Chemical Senses. Vol. 21, pp 323-334, 1996.). The results are shown in Table 3 below.

The % reduction of malodor was calculated by:

% Reduction=(Mean LMS Control−Mean LMS Test)/Mean LMS Control

TABLE 3
Percent reduction of axilla malodor:

| | % Reduction |
|---|---|
| | of Axilla malodor |
| 100% citronellyl methylcrotonate (Sinodor) | 51.0 |
| 80% compound 2 | 73.5 |
| 20% citronellyl methylcrotonate | |
| 50% compound 2 | 67.6 |
| 50% citronellyl methylcrotonate | |
| 20% compound 2 | 57.6 |
| 80% citronellyl methylcrotonate | |

### EXAMPLE 4

### Cyclohexylidene-o-tolyl-acetonitrile

The mixture of cyclohexanone (44.7 g, 0.46 mol) and 2-methyl benzylcyanide (41.9 g, 0.32 mol) is treated with KOH (3.58 g, 0064 mol) and the resulting mixture is heated to 120° C. (oilbath temperature 140° C.) for 2 h with concomitant distillation of H₂O as an azeotrope with cyclohexanone. The temperature is raised to 130° C. (oilbath temperature 150° C.) for 30 min, before cooling to 60° C. and dilution of the viscous mixture with toluene (50 ml). Standard aqueous workup is effected with toluene, H₂O, sat. aq. Na₂CO₃-solution and brine. The organic layer is dried over MgSO₄ and the volatiles removed in vacuo. The crude product (67 g), which crystallizes upon standing, is subjected to a short path distillation at 0.05 mbar/120-127° C. to yield 42.6 g of crystalline product which is recrystallized three times from hexane. From this, 33.6 g of white crystalline product is obtained (50% yield), m.p. 65.2-66.0° C.

Odour description: fruity, rosy, Lychee, Palmarosa, Rosacetol.

¹³C-NMR (100 MHz, CDCl₃): 162.6 (s), 136.9 (s), 133.01 (s), 130.4 (d), 129.9 (d), 128.6 (d), 126.1 (d), 117.9 (s), 106.5 (s), 34.6 (t), 31.3 (t), 28.1 (t), 27.7 (t), 25.9 (t), 19.6 (q). MS: 211 (64, [M⁺]), 196 (20), 182 (17), 168 (31), 154 (45), 144 (63), 143 (100).

### EXAMPLE 5

### Cyclohexylidene-p-tolyl-acetonitrile

The procedure outlined in example 1 is repeated with cyclohexanone (22.34 g, 0.29 mol), p-tolylacetonitrile (20.96 g, 0.16 mol) and KOH (1.79 g, 0.032 mol). Short path distillation of the crude at 0.05 mbar/115-130° C. followed by recrystallization from hexane yields 21.1 g (63% yield) of product as white crystals, m.p. 50.5-51.3° C.

Odour description: floral, rosy, sweet, vanilla

¹³C-NMR (100 MHz, CDCl₃): 161.3 (s), 138.0 (s), 130.9 (s), 129.2 (d), 129.0 (d), 118.7 (s), 107.5 (s), 35.2 (t), 31.2 (t), 28.0 (t), 27.8 (t), 25.8 (t), 21.1 (q). MS: 211 (21, [M⁺]), 196 (5), 182 (3), 168 (9), 154 (17), 143 (100).

1. A composition comprising
a) at least one compound of formula (A) 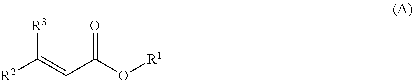
wherein
R′ is linear or branched C₁-C₁₂ alkyl, linear or branched C₃-C₁₂ alkenyl, C₆-C₁₁ aryl, C₇-C₁₂ arylalky, or C₇-C₁₂ arylalkyl substituted with at least one O or N atom;
R² is linear or branched C₁-C₁₀ alkyl, C₆-C₁₀ aryl, C₇-C₁₀ alkoxyaryl, C₄-C₁₀ alkoxycarbonyl;
R³ is hydrogen or methyl; and
R² and R³ are either in the E or Z configuration with respect to the ester group;
and
b) at least one compound of formula (B) 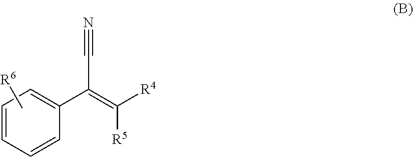
wherein
R⁴ and R⁵ are independently H, linear or branched C₁ to C₈ alkyl, or linear or branched C₂ to C₈ alkenyl, with the proviso that at least one of the residues R⁴ and R⁵ is not hydrogen; or
R⁴ and R⁵ form together with the carbon atom to which they are attached a 5 or 6 membered carbocycle, the carbocycle is optionally substituted with one methyl group;
R⁶ is H, methyl, ethyl, propyl or isopropyl; and
the sum of carbon atoms of the compound of formula (B) is from 11 to 20.

2. A composition according to claim 1 wherein the compound of formula (A) is selected from the group consisting of:
citronellyl methylcrotonate,
geranyl crotonate,
dihexyl fumarate,
benzyl cinnamate,
phenyl cinnamate and
2-ethyl-hexyl-para-methoxy-cinnamate.

3. A composition according to claim 1 wherein the compound of formula (B) is selected from the group consisting of:
3-methyl-2-phenylbut-2-enenitrile,
3-methyl-2-phenylpent-2-enenitrile,
2-phenyloct-2-enenitrile,
2-cyclohexylidene-2-o-tolylacetonitrile,
2-cyclohexylidene-2-p-tolylacetonitrile and
2-cyclohexylidene-2-phenylacetonitrile.

4. A fragrance composition comprising
a) a composition according to claim 1; and
b) at least one odorant.

5. A consumer product comprising an effective amount of a composition according to claim 1.

6. A consumer product according to claim 7 wherein the consumer product is selected from the group consisting of: household products, personal care products and cosmetics.

7. A method of removing malodor from the air or from surfaces, comprising the step of: applying thereto an effective amount of a composition according to claim 1.

8. A method of removing malodor from the air or from surfaces, comprising the step of: applying thereto an effective amount of a composition according to claim 2.

9. A method of removing malodor from the air or from surfaces, comprising the step of: applying thereto an effective amount of a composition according to claim 3.

10. A composition according to claim 2 wherein the compound of formula (B) is selected from the group consisting of:
3-methyl-2-phenylbut-2-enenitrile,
3 -methyl-2-phenylpent-2-enenitrile,
2-phenyloct-2-enenitrile,
2-cyclohexylidene-2-o-tolylacetonitrile,
2-cyclohexylidene-2-p-tolylacetonitrile and
2-cyclohexylidene-2-phenylacetonitrile.
